(19) United States
(12) Patent Application Publication
(10) Pub. No.: US 2024/0218415 A1
(43) Pub. Date: Jul. 4, 2024

(54) ENZYME MUTANTS DIRECTLY ATTACHED TO A NANOGAP DEVICE

(71) Applicant: Universal Sequencing Technology Corporation, Canton, MA (US)

(72) Inventors: Peiming Zhang, Gilbert, AZ (US); Sanjay B. Hari, Sharon, MA (US); Kisup Chung, Mill Creek, WA (US); Ming Lei, Sharon, MA (US)

(73) Assignee: Universal Sequencing Technology Corporation, Canton, MA (US)

(21) Appl. No.: 17/907,111

(22) PCT Filed: Mar. 25, 2021
(86) PCT No.: PCT/US2021/024237

§ 371 Date: Sep. 23, 2022

Related U.S. Application Data
(60) Provisional application No. 62/994,712, filed on Mar. 25, 2020.

(51) Int. Cl. C12Q 1/00

(52) U.S. Cl. CPC C12Q 1/001

(57) ABSTRACT

This invention is related to a nanogap device for the electronic sensing of biomolecules.

### CROSS REFERENCE

This application claims the benefit of U.S. Provisional Patent Application No. 62/994,712, filed Mar. 25, 2020, which is incorporated by reference herein in its entirety.

### FIELD

The present invention is related to a nanodevice for sensing and sequencing biomolecules. More specifically, the invention provides devices, methods, and compositions of matter for the construction of a protein-bridged nanogap device.

### BACKGROUND OF THE INVENTION

One can measure the conductance of a single protein molecule when connecting it to two electrodes separated by a distance of several nanometers. Such an arrangement has achieved using a Scanning Tunneling Microscope (STM) by functionalizing its metal tip and metal substrate with ligands or antigens that recognize their respective cognate proteins.¹ Moreover, the said STM setup could sense biochemical reactions of an enzyme, for instance, Φ29 DNA polymerase from resistive pulses.² These scientific discoveries strongly suggest the possibility of developing an electronic technique to detect the conformational movements of enzymes by measuring electric signals. However, a covalent connection between electrodes and protein makes contact more stable than the non-covalent ones, resulting in improved current flows.

### BRIEF DESCRIPTION OF THE DRAWINGS

FIG. 1 illustrates an electronic nanodevice for monitoring the activities of an enzyme.

FIG. 2 shows a crystal structure of Φ29 DNA polymerase (PDB #1XHX) with its structural regions and cysteine residues indicated.

FIG. 3 illustrates the mutation sites in a wild type Φ29 DNA polymerase in this invention, based on a crystal structure of Φ29 DNA polymerase complexed with primer-template DNA and incoming nucleotide substrates (PDB #: 2PYL).

FIG. 4 shows the process of fabricating a nanogap.

FIG. 5 shows the process of fabricating an array of vertical nanogaps.

FIG. 6 shows the structure of the cysteine mutant of Φ29 DNA polymerase in this invention, based on a crystal structure of Φ29 DNA polymerase complexed with primer-template DNA and incoming nucleotide substrates (PDB #: 2PYL).

FIG. 7 shows the structure of the selenocysteine mutant of Φ29 DNA polymerase in this invention, based on a crystal structure of Φ29 DNA polymerase complexed with primer-template DNA and incoming nucleotide substrates (PDB #: 2PYL).

FIG. 8 shows the structure of the 4-(azidomethyl)-L-phenylalanine mutant of Φ29 DNA polymerase in this invention, based on a crystal structure of Φ29 DNA polymerase complexed with primer-template DNA and incoming nucleotide substrates (PDB #: 2PYL).

FIGS. 9a and 9b illustrate (a) the reaction of azide in 4-(azidomethyl)-L-phenylalanine mutant with the triphenylphosphine ester in the monolayer coated on the electrodes; (b) the direct connection of the protein to electrodes through the formation of amide bonds to bridge the nanogap.

FIG. 10 shows the process of fabricating a nanogap using a thermal chemical lithography method (TCNL).

### SUMMARY OF THE INVENTION

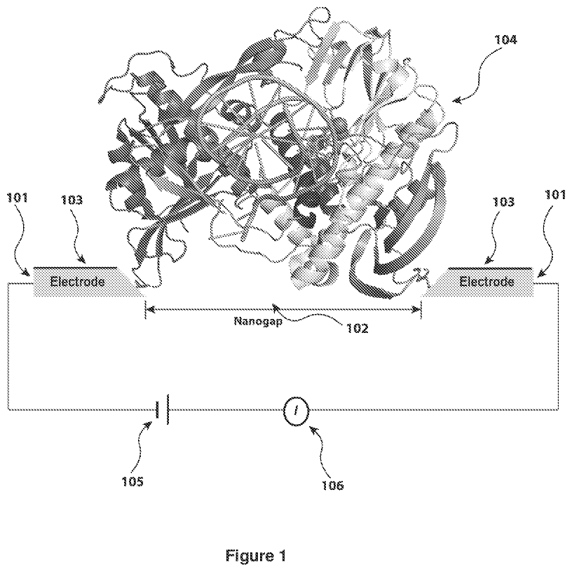

This invention provides an electronic device to monitor the activities of an enzyme directly for sensing a biomolecule. As shown in FIG. 1, the said device comprises a circuit with two electrodes (101) separated by several nanometers to form a nanogap (102). Each electrode is passivated with a dielectric layer (103) except its wedged end. An enzyme (104), which is a mutant of its wild type, is directly attached to the electrodes covalently, bridging the nanogap. The covalent attachment reduces the ohmic resistance, compared to the mentioned above non-covalent contact. The activities of the enzyme can be monitored by recording the electrical signals with an electric signal recording device (105) under a bias (106).

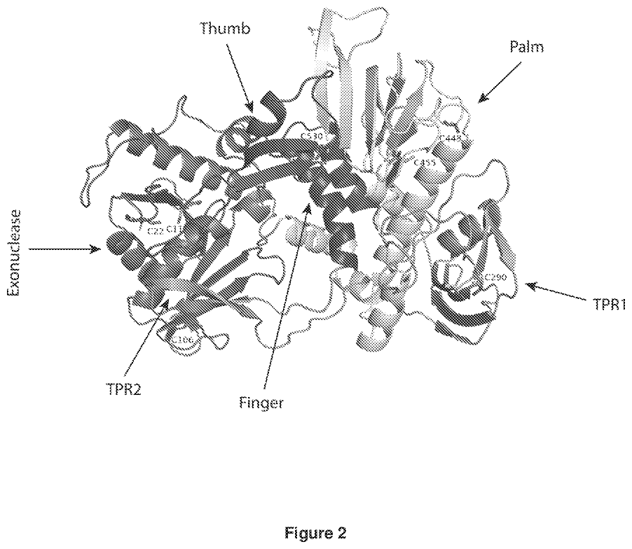

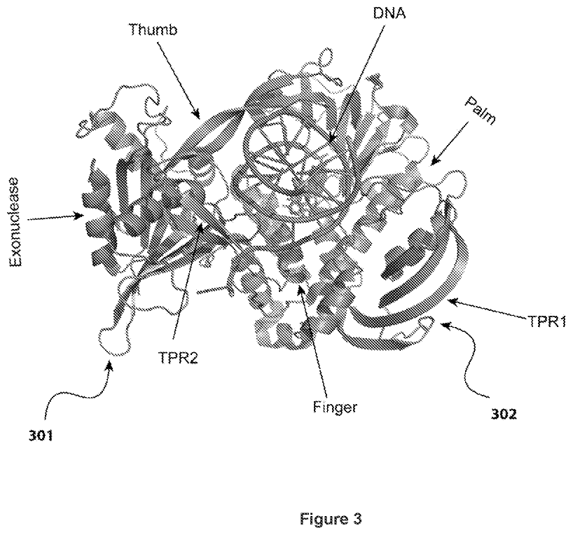

This invention also provides an enzyme mutant with two sites mutated to bear a functional group for the attachment to electrodes without affecting its natural functions. The enzyme can be a DNA polymerase, an RNA polymerase, a DNA helicase, a DNA ligase, a DNA exonuclease, a reverse transcriptase, an RNA primase, a ribosome, a sucrase, lactase, either natural, mutated, synthesized, and a combination thereof. The enzyme can also be replaced by a receptor, a ligand, an antigen, and an antibody, etc., either natural, mutated, or synthesized. Bacteriophage Φ29 DNA polymerase is chosen as an example to demonstrate the merits and novelties of the invention presented in this disclosure. In general, the same principle is applicable to other enzymes. The Φ29 DNA polymerase is an enzyme with high processivity and strand displacement capability to synthesize DNA efficiently.³ It also has a higher fidelity than other polymerases⁴ due to its high nucleotide insertion discrimination values (10⁴-10⁶)⁵ and to the 3′ to 5′ exonuclease activity to proofread polymerization errors.^{6,7} All of these are attributed to the uniqueness of its structure. Based on its crystal structure,⁸ Φ29 DNA polymerase comprises five structural subdomains—exonuclease, TPR1 and TRP2, palm, thumb, finger, respectively (FIG. 2) The palm, thumb, and fingers can be analogous to a semi-open right hand. There are also two insertions specifically present in the protein-primed DNA polymerases subgroup called Terminal Protein Regions 1 (TPR1) and 2 (TPR2). The TPR1 subdomain is involved in the interaction with the terminal protein (TP) for the protein primed initiation. The TPR2, thumb, and palm subdomains form an internal ring-like structure that encircles the upstream duplex DNA at the polymerization active site, providing the enzyme with its inherent high processivity. The TPR2, palm and fingers subdomains, together with the exonuclease domain, form a tunnel that wraps the downstream template strand. The narrow dimensions of this tunnel preclude the dsDNA binding, compelling the melting of the two strands to allow the template to reach the active site and providing to the polymerase the strand displacement capacity. It is well known that the thiol side chain of cysteine reacts with metal surfaces. The Φ29 DNA polymerase has seven cysteine residues, but they are situated in the interior of the protein (shown in FIG. 2), preventing them from reacting with the metal electrodes effectively. In this invention, the Φ29 DNA polymerase mutant contains two amino acid mutations in loops of both exonuclease and TPR1 domains (Site 301 and 302, FIG. 3). The mutation would not affect the biochemical functions of the enzyme, and the two mutation sites are separated in a distance able to bridge the said nanogap.

This invention provides a nanodevice for sensing and sequencing of biomolecules, for example, nucleic acids, proteins, polysaccharides, but not limited to them, either natural, synthesized, modified, and a combination thereof.

### DETAILED DESCRIPTION OF THE INVENTION

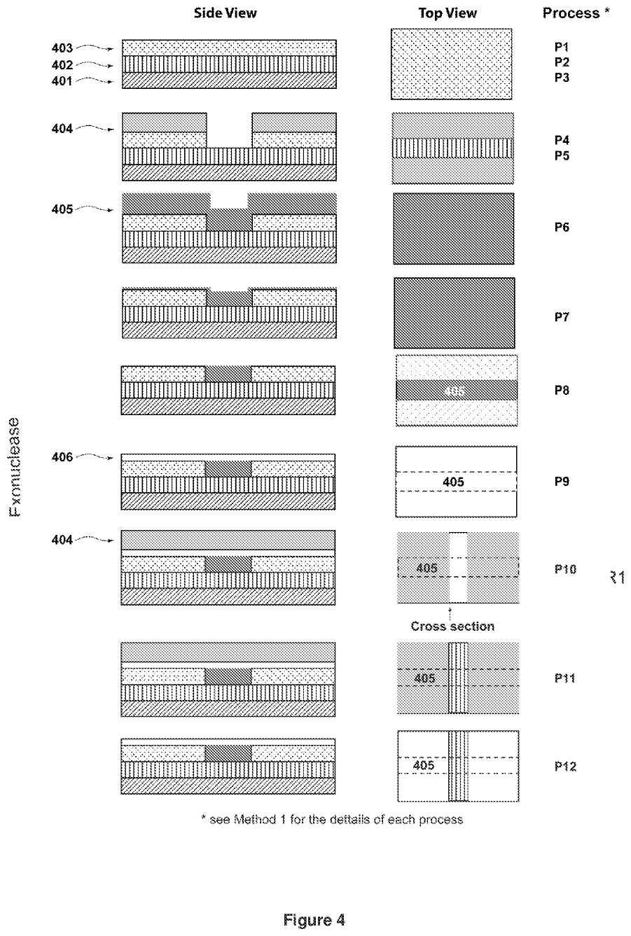

In one embodiment, this invention provides a nanogap formed by two nano-electrodes separated by a distance ranging from 3 nm to 20 nm. The ends of these two electrodes are wedged at their nanogap side, and their top surfaces are covered by a dielectric layer and/or a monolayer of chemical passivation molecules. The process of fabricating a nanogap is shown in FIG. 4 and described in Method 1 in detail.

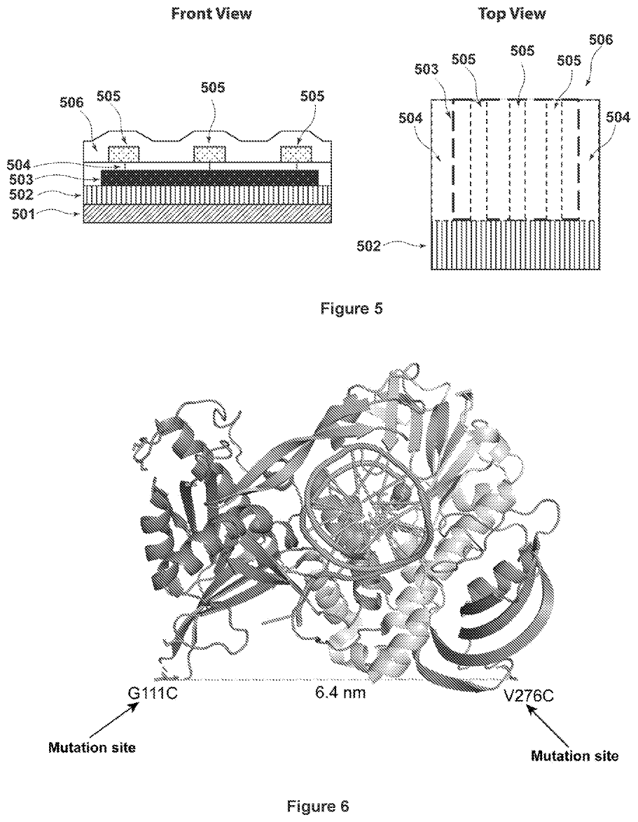

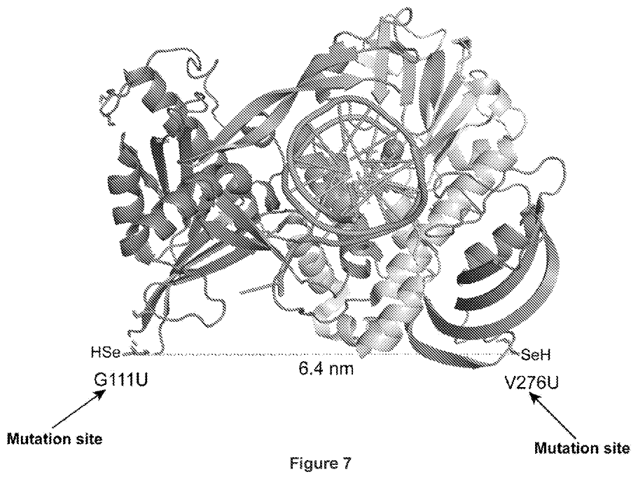

In another embodiment, this invention provides an array of electrodes vertically separated from a single bottom electrode by a dielectric layer (FIG. 5). This type of format allows for a higher packaging density of nanogaps. Besides, all of the top electrodes have the same electrical polarity, which provides a means to prevent the charged molecules from a lateral contact cross-linking between top electrodes. The lateral distance between the top electrodes is comparable to or larger than the vertical gap size, from several nanometers (nm) to micrometers (um) and millimeters (mm), with basically no upper limit.

In some embodiments, the proteins used to bridge the nanogap are C to X mutants of wild type Φ29 DNA polymerase with between 2 and 7 cysteines, inclusive, mutated. The polymerase engineering process is described in Method 3. As indicated in Table 1, the mutants with C22A and C290A (M-2), as well as C22A. C290A, and C455V (M-4) have the same activities as the wild type. The other mutants are less active compared to the wild type.

TABLE 1
Cysteine mutagenesis of Φ29 DNA polymerase

| | Cysteine (C) | | | | | | | |
|---|---|---|---|---|---|---|---|---|
| Mutant | C11 | C22 | C106 | C290 | C448 | C455 | C530 | Activity |

| | | | | | | | | |
|---|---|---|---|---|---|---|---|---|
| Amino | M-1 | A/V/ | | | | | | unstable |
| acid | | G/I/M/T | | | | | | |
| (X) | M-2 | | A | A | | | | ≈WT |
| | M-3 | | A | A | V | | | Low yield |
| | M-4 | | A | A | | V | | ≈WT |
| | M-5 | | A | A | V | V | | Low yield |
| | | | | | | | | low activity |
| | M-6 | | A | A | | V | V | Low yield |
| | | | | | | | | low activity |
| | M-7 | | A | A | V | V | V | Low yield |
| | | | | | | | | low activity |

In some embodiments, the protein used to bridge the nanogap is a mutant of wild-type Φ29 DNA polymerase with mutations of G111C and V276C (FIG. 6). The newly introduced cysteines are situated at the loops of the protein, separated by a distance of ˜6.4 nm. They can be accessed more quickly compared to native cysteines. The thiol groups of the two engineered cysteines react respectively with metal electrodes to bridge a nanogap covalently.

In some embodiments, the metal surfaces are passivated by ω-mercapto PEG (SR-1, shown below) to prevent non-specific adsorption on the electrodes after the enzyme is attached:

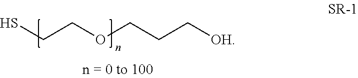

In some embodiments, the protein is a mutant of wild-type Φ29 DNA polymerase with mutations of G111U and V276U (U is selenocysteine). The Se—Au bond is more stable as compared to the S—Au one in the analogous SAMs, albeit both having a similar total probability of charge carrier tunneling.⁹ Selenocysteine has a pKa of ˜5.2, which means that its side-chain selenol is deprotonated at the physiological pH.¹⁰

In one embodiment, this invention provides a method to synthesize a chemical reagent (CR-1) for the formation of a monolayer on the metal electrodes (Scheme 1, see Method 4 for details). The triphenylphosphaneyl ester of CR-1 reacts with an azido function to form an amide bond.¹¹

In another embodiment, this invention provides a method to synthesize a chemical reagent (CR-2) in a similar way as described in Method 4 for the formation of a monolayer on the metal electrodes (Scheme 2). The triphenylphosphaneyl ester of CR-2 reacts with an azido function to form an amide bond.

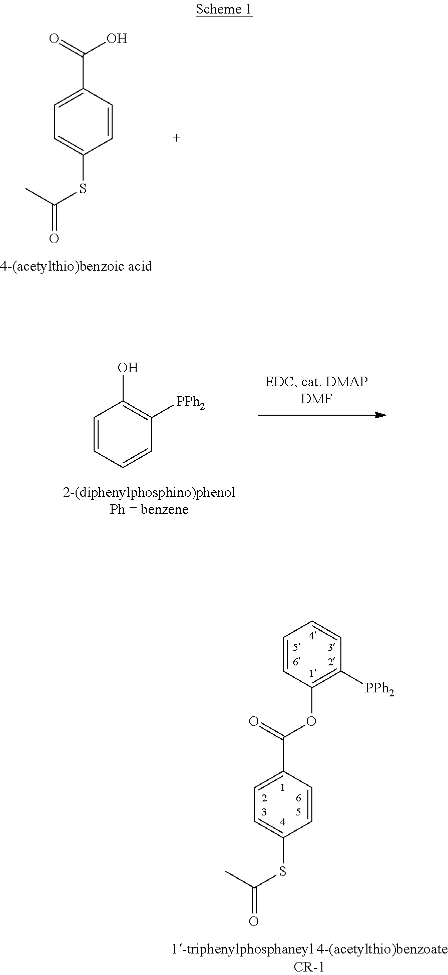

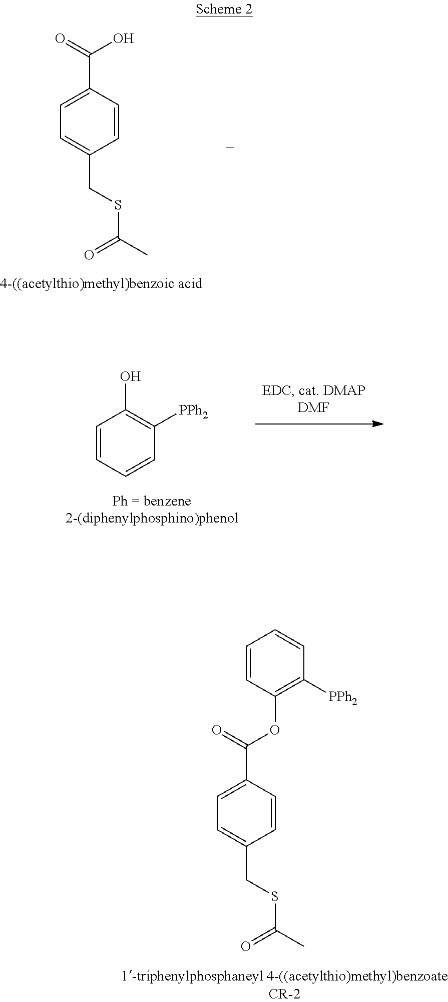

In some embodiments, the surfaces of electrodes are covered by a monolayer of CR-1, CR-2, or a mixture of SR-1 with CR-1 or CR-2. This invention provides a method to form the said monolayers (Method 5).

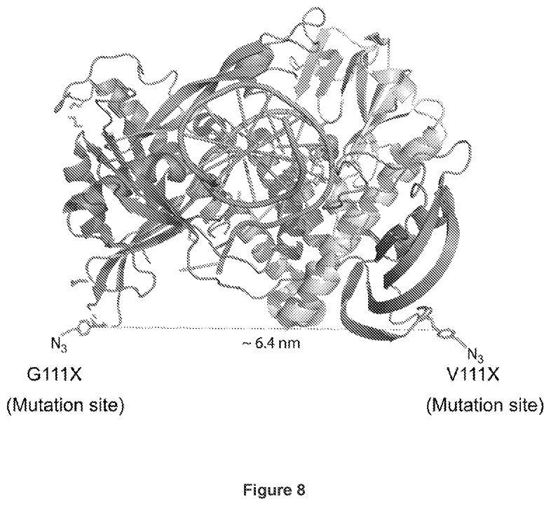

In one embodiment, this invention provides a mutated Φ29 DNA polymerase of G111X and V276X (X is 4-(Azidomethyl)-L-phenylalanine) (FIG. 8) for bridging the said nanogaps. The mutated protein is expressed by a method as described in Method 6.

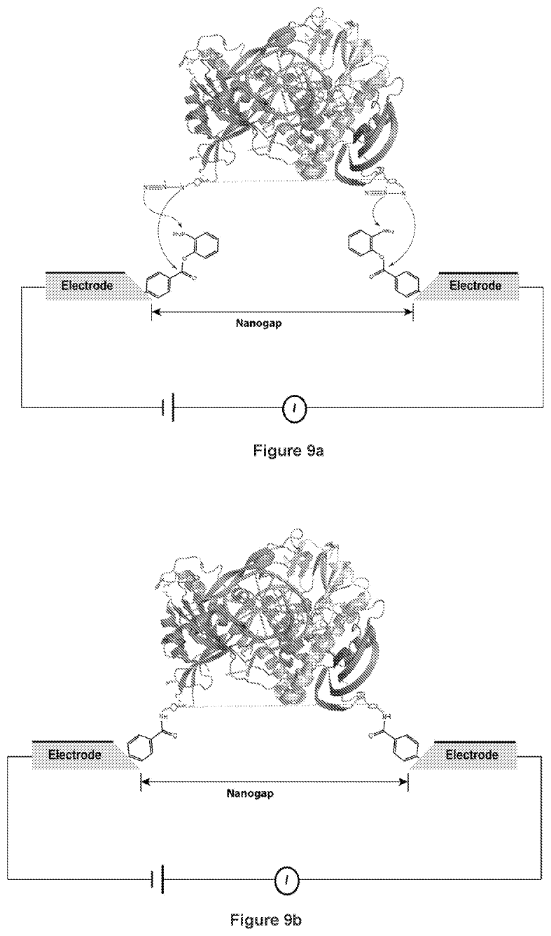

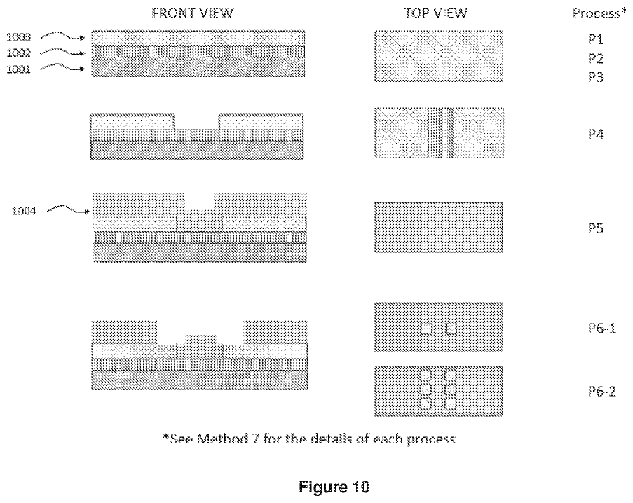

In some embodiments, this invention provides a method to attach the azido mutant to electrodes coated with CR-1 or CR-2 for bridging the nanogap through a Staudinger reaction. As shown in FIG. 9a, the azide reacts with triphenylphosphaneyl ester through a traceless Staudinger reaction to form an amide bond shown in FIG. 9b to connect the protein to electrodes.

In another embodiment, an unrelated protein (non-limiting examples include Smt3 from Saccharomyces cerevisiae and glutathione-S-transferase from Schistosoma japonicum) is inserted genetically between two secondary structure elements of Φ29 DNA polymerase (including but not limited to, residues K110 and G111, K150 and E151, and Y156 and K157). Such a protein that retains catalytic activity could be used in conjunction with the embodiments described above to bridge an elongated gap, which would be too wide for wild-type Φ29 DNA polymerase.

In another embodiment, an unrelated protein (non-limiting examples including Smt3 from Saccharomyces cerevisiae and glutathione-S-transferase from Schistosoma japonicum) is inserted at the N-terminus Φ29 DNA polymerase and connect by a rigid peptide (one non-limiting example is the sequence PAPAP). Such a protein that retains catalytic activity could be used in conjunction with the embodiments described above to bridge an elongated gap, which would be too wide for wild-type Φ29 DNA polymerase.

In one embodiment, this invention provides a single nanogap or multiple nanogaps on a conductive layer using a thermal chemical lithography method (TCNL)^{12, 13}, see Method 7.

### Methodology

Method 1 is related to the workflow delineated in FIG. 4, producing said nanogaps by following the procedure below.
P1: prepare semiconductor or insulating (glass) substrate (401).
P2: deposit an insulating layer (402) of SiNₓ, SiOₓ, or other dielectric materials by chemical vapor deposition (CVD), atomic layer deposition (ALD), physical vapor deposition (PVD), molecular vapor deposition (MVD), Electroplating, or Spin Coating. The preferred method is plasma-enhanced CVD (PECVD) or low-pressure CVD (LPCVD).
P3: deposit another insulating layer (403) of SiNₓ, SiOₓ, or any dielectric materials by chemical vapor deposition (CVD), atomic layer deposition (ALD), physical vapor deposition (PVD), molecular vapor deposition (MVD), Electroplating, or Spin Coating. The preferred method is plasma-enhanced CVD (PECVD) or low-pressure CVD (LPCVD).
P4: carry out a process of electrode line patterning by EBL with a dose of 10,000˜500,000 uC/cm², followed by photo lithograph using a photoresist (404) as a mask
P5: carry out the line etching using RIE or IBE, followed by Reactive Ion Etching (RIE) or Ion Beam Etching (IBE), stopping on the insulating layer 402 or with little over-etching, followed by removing the photoresist mask.
P6: deposit an electrode layer (405) of a conductive material, such as Au, Pt, Pd, W, Ti, Ta, TiNx, TaNx, Al, Ag, or other metal composites, and/or common HK/MG materials used in semiconductor. It can also be a combination of two or more layers to provide good adhesion and electrical/chemical properties. It can be prepared by the methods mentioned in P2, and the most preferred method is ALD.
P7: carry out a process of chemical mechanical polishing, followed by planarization (CMP).
P8: complete a CMP touch-up.
P9: deposit a dielectric layer (406) of SiNₓ, SiOₓ, AlₓO_{y}, HfOₓ, or other dielectric materials by chemical vapor deposition (CVD), atomic layer deposition (ALD), physical vapor deposition (PVD), molecular vapor deposition (MVD), electroplating, or spin coating, etc. The preferred method is ALD.
P10: carry out a process of electrode gap patterning using EBL with a dose of 10,000˜500,000 uC/cm², followed by photolithography.
P11: carry out a process of gap etching using RIE, or IBE, stopping on the insulating layer 402 or with little over-etching.
P12: strip the photoresist.
P13. lift-off interconnects & pad patterning, followed by deletion.

Method 2 is related to the workflow delineated in FIG. 5, producing an array of said nanogaps by following the procedure below.
P1: prepare a semiconductor or insulating (e.g., glass) substrate (501).
P2: deposit an insulating layer (502) of SiNₓ, SiOₓ, AlₓO_{y}, HfOₓ, or other dielectric materials by chemical vapor deposition (CVD), atomic layer deposition (ALD), physical vapor deposition (PVD), molecular vapor deposition (MVD), Electroplating, or Spin Coating. The preferred method is plasma-enhanced CVD (PECVD) or low-pressure CVD (LPCVD).
P3 & P4: deposit a bottom electrode layer (503) of a common metallic, conductive material, such as Au, Pt, Pd, W, Ti, Ta, TiNₓ, TaNₓ, Al, Ag, other metals, metal composites, and/or common HK/MG materials used in the semiconductor industry by the methods mentioned in P2. The preferred method is ALD. Furthermore, the electrode layer can be patterned by a line patterning method (EBL, EUV, DUV, Contact Mask) to have the width of an electrode larger than 1 nm.
P5: deposit a dielectric layer (504) to function as a nanogap of SiNₓ, SiOₓ, AlₓO_{y}, HfOₓ, or other dielectric materials by CVD, ALD, PVD, MVD, Electroplating, or Spin Coating (504). The preferred method is ALD. The gap size is generally comparable to the diameter of the protein molecules.
P6 & P7: fabricate the top electrode array (505) comprising a conductive material, Au, Pt, Pd, W, Ti, Ta, TiNₓ, TaNₓ, Al, Ag, other metals, metal composites, and/or common HK/MG materials used in the semiconductor industry, by the method mentioned in P2. The preferred method is ALD. The fabrication of electrode arrays is carried out using the line patterning—EBL, EUV, DUV, or Contact Mask, and the etching method. The width and thickness of each electrode are larger than 1 nm.
P8: deposit a cap dielectric layer of SiNₓ, SiOₓ, AlₓO_{y}, HfOₓ, or other dielectric materials by CVD, ALD, PVD, MVD, Electroplating, or Spin Coating. The preferred method is ALD.

Method 3: Codons for cysteine residues are mutated to those for other residues (including but not limited to alanine, valine, serine, glycine, and leucine) by site-directed mutagenesis,¹⁴ using a plasmid harboring a gene for Φ29 DNA polymerase as a template. All mutations are verified by dideoxy (Sanger) sequencing. Plasmids harboring the desired mutant genes are transformed into BL-21(DE3) cells. Liquid cultures are grown, and the expression of the protein is induced with IPTG. After growth for 3 h at 30° C., the cells are harvested, lysed, and the recombinant protein is purified by Ni-NTA chromatography. The protein is further purified using a heparin column. Proteins are stored at −80° C. for later use. Plasmids harboring genes for protein mutants that display sufficient expression and catalytic activity are used as templates for further rounds of site-directed mutagenesis.

Method 4: To a solution of 4-(acetylthio)benzoic acid in anhydrous DMF, 1-Ethyl-3-(3-dimethylaminopropyl)carbodiimide (EDC), and a catalytic amount of dimethylaminopyridine (DMAP) are added. The solution is stirred at 0° C. for 30 minutes, followed by the addition of a solution (2-hydroxyphenyl)diphenylphosphine in anhydrous DMF and stirred overnight. Then, the solvent is removed by rotary evaporation, and the residue was purified by flash column chromatography using 5% methanol in dichloromethane to give the desired product.

Method 5: A solution of CR-1 or CR-2 in ethanol is first treated with pyrrolidine for one hour to remove the acetyl protecting group under nitrogen. Then, the solution is added to the nanogap substrate and incubated for one hour, followed by rinsing the substrate with ethanol.

Method 6: Codons at specific positions (including but not limited to 33, 111, 276, and 369) of Φ29 DNA polymerase are mutated to TAG by site-directed mutagenesis,¹⁴ using a plasmid harboring a gene for Φ29 DNA polymerase as a template. All mutations are verified by dideoxy (Sanger) sequencing. Plasmids harboring the desired mutant genes are co-transformed with pEVOL-pAzF¹⁵ into BL-21(DE3) cells. Liquid cultures are grown, and the expression of the protein is induced with IPTG and arabinose. Further growth and protein expression are performed, as described in Method 3.

Method 7 is related to the workflow delineated in FIG. 10 to produce a nanosensor using a thermal chemical lithography method (TCNL).
P1: prepare a semiconductor or insulating (e.g., glass) substrate (1001).
P2: deposit an insulating layer (1002) of SiNₓ, SiOₓ, AlₓO_{y}, HfOₓ, or other dielectric materials by chemical vapor deposition (CVD), atomic layer deposition (ALD), physical vapor deposition (PVD), molecular vapor deposition (MVD), Electroplating, or Spin Coating. The preferred method is plasma-enhanced CVD (PECVD) or low-pressure CVD (LPCVD).
P3: deposit a bottom electrode layer (1003) of a common metallic, conductive material, such as Au, Pt, Pd, W, Ti, Ta, TiNₓ, TaNₓ, Al, Ag, other metals, metal composites, and/or common HK/MG materials used in the semiconductor industry by the methods mentioned in P2. The preferred method is ALD.
P4: pattern the electrode layer (1003) by a line patterning method (EBL, EUV, DUV, Contact Mask) followed by etching the electrode larger to create a gap with a predefined width.
P5: spin-coat a protective cap layer (1004) that is temperature-responsive (compatible with TCNL) such as but not limited to polyphthalaldehyde polymer (PPA).
P6-1: thermochemically remove a predefined volume of the cap layer (1004), exposing the desired area of single paired electrodes (1003).
P6-2: Thermochemically remove a predefined volume of the cap layer (1004), exposing multiple areas of multi-paired electrodes (1003).

The following are some claimable critical points of this invention:
1. A system for identification, characterization, or sequencing of a biopolymer comprising,
   (a) a nanogap formed by a first electrode and a second electrode separated by a distance ranging from 3 nm to 20 nm (planar nanogap) or by a dielectric insulation layer with a thickness between 2 nm to 20 nm (vertical nanogap);
   (b) a protein mutant bearing two functional groups separated by a distance comparable to or larger than the nanogap size for bridging the said nanogap by the first one to react with the first electrode and the second one to the second electrode covalently;
   (c) a bias voltage that is applied between the first electrode and the second electrode;
   (d) a device that records the electric signals generated by the said protein while it carries outs chemical reactions; and
   (e) software for the data analysis.
2. A method for monitoring the activities of an enzyme:
   (a) Providing a nanogap formed by a first electrode and a second electrode separated by a distance ranging from 3 nm to 20 nm (planar nanogap) or by a dielectric insulation layer with a thickness between 2 nm to 20 nm (vertical nanogap);
   (b) Providing an enzyme mutant that bears at least two functional groups ether from the side chain of natural or unnatural amino acids, separated by a distance comparable to or larger than the nanogap size for the attachment to electrodes;
   (c) Bridging the said nanogap by attaching the enzyme through the first functional group to react with the first electrode and the second functional group to the second electrode covalently;
   (d) Applying a bias voltage between the first electrode and the second electrode;
   (e) Recording the electric signals generated by the enzyme reacting with its substrates; and
   (f) Providing software for data analysis.
3. A method for identification, characterization, or sequencing of a biopolymer:
   (a) Providing a nanogap formed by a first electrode and a second electrode separated by a distance ranging from 3 nm to 20 nm (planar nanogap) or by a dielectric insulation layer with a thickness between 2 nm to 20 nm (vertical nanogap);
   (b) Providing a polymerase mutant that bears at least two functional groups ether from the side chain of natural or unnatural amino acids, separated by a distance comparable to or larger than the nanogap size for the attachment to electrodes;
Bridging the said nanogap by attaching the enzyme through the first functional group to react with the first electrode and the second functional group to the second electrode covalently;
   (c) Applying a bias voltage between the first electrode and the second electrode;
   (d) Recording the electric signals generated by the enzyme reacting with its substrates; and
   (e) Providing software for data analysis.
4. A method to fabricate nanogaps and an array of nanogaps using a thermal chemical lithography method (TCNL).
5. A method to form a monolayer on the surface of electrodes to prevent the non-specific adsorption of biological molecules.
6. An approach to synthesize chemical reagents CR-1 and CR-2 for the formation of a mixed monolayer.
7. A method to bridge the said nanogap via a reaction of CR-1 or CR-2 with a protein mutant by forming the amide bond.

General Remarks: Unless defined otherwise, all technical publications, patents, and other documents mentioned herein are incorporated by reference in their entirety, and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will readily appear to those skilled in the art. The invention in its broader aspects is therefore not limited to the specific details, representative device, apparatus and method, and illustrative example shown and described. Accordingly, departures may be made from such details without departing from the spirit of the applicant's general inventive concept.

### CITED REFERENCES

1. Zhang, B.; Song, W.; Pang, P.; Lai, H.; Chen, Q.; Zhang, P.; Lindsay, S., Role of contacts in long-range protein conductance. PNAS 2019, 116, 5886-5891.
2. Zhang, B.; Deng, H.; Mukherjee, S.; Song, W.; Wang, X.; Lindsay, S., Engineering an Enzyme for Direct Electrical Monitoring of Activity. ACS Nano 2019, 14, 1360-1368.
3. Blanco, L.; Bernad, A.; Lharo, J. M.; Martin, G.; Garmendia, C.; Salasn, M., Highly Efficient DNA Synthesis by the phi29 DNA Polymerase. J. Biol. Chem. 1989, 15, 8935-8940.
4. de Paz, A. M.; Tyo, K. E J.; Cybulski, T. R.; Marblestone, A. H.; Church, G. M.; Zamft, B. M.; Boyden, E. S.; Kording, K. P., High-resolution mapping of DNA polymerase fidelity using nucleotide imbalances and next-generation sequencing. Nucleic Acids Research 2018, 46, e78-e78.
5. Esteban, J. A.; Salas, M.; Blanco, L., Fidelity of Φ29 DNA Polymerase. J. Biol. Chem. 1993, 268, 2713-2726.
6. Blanco, L.; Salas, M., Characterization of a 3′-5′ exonuclease activity in the phage phi29-encoded DNA polymerase. Nucleic Acids Research 1985, 13, 1239-1249.
7. Garmendia, C.; Bernad, A.; Estebane, J. A.; Blanco, L.; Salad, M., The Bacteriophage phi29 DNA Polymerase, a Proofreading Enzyme. J. Biol. Chem. 1992, 267, 2594-2599.
8. Kamtekar, S.; Berman, A. J.; Wang, J.; Lazaro, J. M.; de Vega, M.; Blanco, L.; Salas, M.; Steitz, T. A., Insights into strand displacement and processivity from the crystal structure of the protein-primed DNA polymerase of bacteriophage phi29. Molecular cell 2004, 16, 609-618.
9. Ossowski, J.; Wächter, T.; Silies, L.; Kind, M.; Noworolska, A.; Blobner, F.; Gnatek, D.; Rysz, J.; Bolte, M.; Feulner, P.; Terfort, A.; Cyganik, P.; Zharnikov, M., Thiolate versus Selenolate: Structure, Stability, and Charge Transfer Properties. ACS Nano 2015, 9, 4508-4526.
10. Mousa, R.; Notis Dardashti, R.; Metanis, N., Selenium and Selenocysteine in Protein Chemistry. Angewandte Chemie International Edition 2017, 56, 15818-15827.
11. Senapati, S.; Biswas, S.; Zhang, P., Traceless Staudinger Ligation for Biotinylation of Acetylated Thiol-Azido Heterobifunctional Linker and Its Attachment to Gold Surface. Current Organic Chemistry 2018, 22, 411-415.
12. Wang, D.; Kodali, V. K.; Underwood II, W. D.; Jarvholm, J. E.; Okada, T.; Jones, S. C.; Rumi, M.; Dai, Z.; King, W. P.; Marder, S. R.; Curtis, J. E.; Riedo, E., Thermochemical Nanolithography of Multifunctional Nanotemplates for Assembling Nano-Objects. Advanced Functional Materials 2009, 19, 3696-3702.
13. Carroll, K. M.; Lu, X.; Kim, S.; Gao, Y.; Kim, H.-J.; Somnath, S.; Polloni, L.; Sordan, R.; King, W. P.; Curtis, J. E.; Riedo, E., Parallelization of thermochemical nanolithography. Nanoscale 2014, 6, 1299-1304.
14. Liu, H.; Naismith, J. H., An efficient one-step site-directed deletion, insertion, single and multiple-site plasmid mutagenesis protocol. BMC Biotechnol 2008, 8, 91.
15. Young, T. S.; Ahmad, I.; Yin, J. A.; Schultz, P. G., An enhanced system for unnatural amino acid mutagenesis in E. coli. J Mol Biol 2010, 395, 361-374.

1. A system for identification, characterization, or sequencing of a biopolymer comprising,
a nanogap formed by a first electrode and a second electrode separated by a nanometer distance on a non-conductive substrate (planar nanogap) or by a dielectric insulation layer with a nanometer thickness (vertical nanogap); and
a protein engineered to bear at least two functional groups separated by a distance comparable to the nanogap size that bridges the said nanogap by covalently attaching to the first electrode through a first functional group of the at least two functional groups and to the second electrode through a second functional group of the at least two functional groups, wherein the two functional groups are different from each other or the same.

2. The system of claim 1, further comprising,
a bias voltage that is applied between the first electrode and the second electrode;
a device that records a current fluctuation caused by the protein as it interacts or performs a biochemical reaction with the biopolymer; and
a software for data analysis that identifies or characterizes the biopolymer or a subunit of the biopolymer.

3. The system of claim 1, wherein the biopolymer is selected from the group consisting of a DNA, an RNA, a protein, a carbohydrate, a polypeptide, an oligonucleotide, a polysaccharide, and their analogs, either natural, synthesized, modified, and a combination thereof.

4. The system of claim 1, wherein the protein is selected from the group consisting of an enzyme, a receptor, a ligand, an antigen, and an antibody, either native, mutated, synthesized, and a combination thereof.

5. The system of claim 4, wherein the enzyme is selected from the group consisting of a DNA polymerase, an RNA polymerase, a DNA helicase, a DNA ligase, a DNA exonuclease, a reverse transcriptase, an RNA primase, a ribosome, a sucrase, lactase, either natural, mutated, synthesized, and a combination thereof.

6. The system of claim 5, wherein the DNA polymerase is a Φ29 DNA polymerase.

7. The system of claim 1, wherein the functional group is selected from the group consisting of thiol, selenol, azide and a combination thereof.

8. The system of claim 1, wherein the protein is a mutant of wild type Φ29 DNA polymerase with mutations selected from the group consisting of (a) C22A and C290A mutations; (b) C22A, C290A, and C455V mutations; (c) G111X and V276X mutations, wherein X is cysteine or selenocysteine or 4-(Azidomethyl)-L-phenylalanine, or a combination thereof; (d) G111C and V276C mutations; (e) G111U and V276U mutations; (f) G111X and V276X mutations, wherein X is 4-(Azidomethyl)-L-phenylalanine; and (g) a combination thereof.

9. The system of claim 1, wherein the end surfaces of the electrodes at the nanogap is configured to functionalize with a 1′-triphenylphosphaneyl 4-(acetylthio)benzoate (CR-1) or a 1′-triphenylphosphaneyl 4-((acetylthio)methyl)benzoate (CR-2) or a thiolated oligo(ethylene glycol) (SR-1) or a thiolated poly(ethylene glycol), or a mixture of the SR-1 with the CR-1 or the CR-2.

10. The system of claim 1, wherein the distance between the two functional groups on the protein is extended by genetically inserting another unrelated protein and/or a peptide into the protein.

11. The system of claim 10, wherein the unrelated protein is a Smt3 from Saccharomyces cerevisiae or a glutathione-S-transferase from Schistosoma japonicum, and the peptide is PAPAP.

12. The system of claim 10, wherein the protein is a Φ29 DNA polymerase, either wild type, mutated or synthesized, and the location for the insertion of the unrelated protein and/or peptide is at the N-terminus or between residues K110 and G111, or K150 and E151, or Y156 and K157, or a combination thereof.

13. (canceled)

14. The system of claim 1, wherein the nanogap size is about 3 nm to 20 nm.

15. The system of claim 1, wherein the ends of the two electrodes in a planar nanogap are substantially wedge-shaped or substantially tapered at the nanogap.

16. The system of claim 1, wherein the top surfaces of the electrodes except the end surfaces at the nanogap are substantially covered by a dielectric layer and/or a monolayer of chemical passivation molecules.

17. The system of claim 16, wherein the passivation molecule comprises a ω-mercapto PEG (SR-1).

18. The system of claim 1, wherein the vertical nanogap comprises an array of nanogaps formed by an array of first electrodes and a single second electrode separated by a dielectric layer.

19. (canceled)

20. The system of claim 1, wherein the two electrodes are fabricated by cutting through a continuous conductive wire using a thermal chemical lithography method (TCNL), and the gap and the electrodes are filled or covered by a layer of TCNL compatible material with a pair of exposed nano-islets across the gap represent the end surfaces of the two electrodes that form the nanogap.

21. The system of claim 20, wherein the TCNL compatible material comprises a polyphthalaldehyde polymer (PPA).

22. The system of claim 20, wherein the nanogap comprises a plurality of nanogaps formed on the same electrode pair with a plurality of exposed nano-islet pairs.

23-44. (canceled)
